(19) United States
(12) Patent Application Publication
(10) Pub. No.: US 2011/0151089 A1
(43) Pub. Date: Jun. 23, 2011

(54) COMPOSITION CONTAINING MAGNESIUM GLUTAMATE WHICH CAN ENHANCE THE SALTINESS OF FOODS AND DRINKS

(76) Inventors: Toshifumi Imada, Kawasaki-shi (JP); Mayumi Ishida, Koshigaya-shi (JP); Rumiko Kawano, Koshigaya-shi (JP); Heii Arai, Koshigaya-shi (JP); Rirei Sou, Kawasaki-shi (JP); Eiichiro Kimura, Kawasaki-shi (JP)

(21) Appl. No.: 12/983,539

(22) Filed: Jan. 3, 2011

Related U.S. Application Data
(63) Continuation of application No. PCT/JP2009/062191, filed on Jul. 3, 2009.

(30) Foreign Application Priority Data
Jul. 4, 2008 (JP) 2008-175709

(51) Int. Cl. A23L 1/22; C07C 229/24

(52) U.S. Cl. 426/534; 562/573

(57) ABSTRACT

A composition which enhances the saltiness and the salty flavor of food or drink, and a method for enhancing the saltiness and the salty flavor of food or drink are described. The composition contains magnesium glutamate, and can be used to improve a decrease in the taking ratio of a food or a drink, which contains sodium chloride in a reduced amount, because of the lack of the salty taste thereof.

This application is a Continuation of, and claims priority under 35 U.S.C. §120 to, International Application PCT/JP2009/062191, filed Jul. 3, 2009, and claims priority therethrough under 35 U.S.C. §119 to Japanese patent application No. 2008-175709, filed on Jul. 4, 2008, the entireties of which are incorporated by reference herein.

### BACKGROUND OF THE INVENTION

1. Field of the Invention

The present invention relates to a composition containing magnesium glutamate which is able to enhance the saltiness, taste, and/or flavor of food and drink, and particularly the composition is able to enhance the saltiness of food and drink which have a reduced sodium chloride content, so that food or drink intake is the same as compared to non-reduced sodium chloride content. Also described are methods for enhancing the saltiness, flavor, and taste of food and drinks, particularly reduced-salt food and drinks, and methods for reducing sodium chloride in a food or drink.

2. Brief Description of the Related Art

Excessive intake of sodium chloride is a cause of high blood pressure and is also considered to be a cause of cerebral apoplexy and various cardiac diseases. Prevention of these diseases is generally accomplished by reducing sodium chloride intake, or using a sodium chloride substitute. However, foods containing a reduced amount of sodium chloride can have reduced appeal in terms of taste and flavor. In addition, it has been reported that people trying to adhere to a reduced sodium foods actually have reduced nutrient intake for nutrients such as fat and protein, which results in decreased energy (see Morris, C. D. Effect of dietary sodium restriction on overall nutrient intake, Am. J. Clin. Nutr., Vol. 65, pp. 687s-691s (1997), for example). The addition of sodium glutamate or spices to reduced-sodium chloride foods has been reported as a method to improve the tastiness and flavor caused by a decrease in sodium chloride (see Japanese Journal of Taste and Smell Research, vol. 13, No. 3, pp 447-450 (2007), for example). However, while the sodium glutamate or spices can broaden the tastiness and overall flavor, it cannot enhance the salty taste or saltiness itself.

On the other hand, it has been reported that a portion of the sodium chloride can be replaced with a sodium chloride substitute such as a potassium salt, ammonium salt, basic amino acid or peptide having a salty taste, and the like. For example, adding potassium chloride by adding carrageenan with potassium chloride can reduce a bitter taste (see Japanese Patent Kokai Publication No. JP-P04-262758A, for example), and fermenting food containing potassium chloride (see Japanese Patent Kokai Publication No. JP-P2007-289145A, for example) has also been reported. In addition, materials have been reported that can enhance the saltiness of sodium chloride, although the material itself has no salty taste. For example, saturated aliphatic monocarboxylic acid having 3 to 8 carbon atoms can be added to a food or drink that contains sodium chloride, in an amount of 0.01 to 1 weight % relative to the weight of the sodium chloride (see Japanese Patent Kokai Publication No. JP-P05-184326A, for example).

### SUMMARY OF THE INVENTION

Adding sodium glutamate to a reduced-salt food can improve the taste and flavor of the food by enhancing the overall good taste; however, enhancement of the salty taste itself is not enough. On the other hand, since sodium glutamate contains sodium, further salt-reduction can be anticipated by using a glutamate other than a sodium salt. However, although the basic taste characteristics of various glutamates have been previously reported, a glutamate, other than sodium glutamate, having a salty taste has not been previously reported.

The inventors have found that magnesium glutamate, although it has no salty taste, has an excellent enhancing effect of the salty taste when mixed with sodium chloride.

According to an aspect of the present invention, there is provided a composition comprising magnesium glutamate that is able to enhance the saltiness, the salty taste, or the flavor of a food or drink. The composition can be used to improve food intake when on a reduced-salt diet, since eating reduced-salt foods usually results in a decrease in food intake due to the unappetizing nature of the food.

According to another aspect of the present invention, there is provided a method for enhancing the saltiness, the salty taste, or the flavor of a food or a drink by adding magnesium glutamate to a food or a drink containing sodium chloride. The amount of the magnesium glutamate that can be added to the food or drink can be 1 to 200 weight % relative to the weight of the sodium chloride in the food or drink. It can also be 5 to 100 weight %, or 10 to 80 weight %, of the sodium chloride in the food or drink.

According to another aspect of the present invention, there is provided a seasoning composition comprising sodium chloride and magnesium glutamate, wherein the amount of magnesium glutmate is 0.5 to 100 weight % relative to the weight of the sodium chloride. The amount of magnesium glutamate can also be 1 to 100 weight %, or even 5 to 100 weight % relative to the weight of the sodium chloride. The seasoning composition can further contain one or more components such as sodium glutamate, potassium glutamate, calcium glutamate, ammonium glutamate, glutamic acid, and other flavorful components based on or derived from nucleic acid.

According to further aspects of the presently disclosed subject matter, there is provided a method for reducing sodium chloride in a food or a drink by substituting a portion of the sodium chloride with magnesium glutamate. The amount of added magnesium glutamate can be 2 to 500 weight % of sodium chloride. The amount of the magnesium glutamate can be 10 to 300 weight % of the sodium chloride or even, 10 to 100 weight % of the sodium chloride.

The composition as described in accordance with the presently disclosed subject matter is able to enhance the saltiness, the salty taste, or the flavor of sodium chloride when added to a food or drink containing sodium chloride, as well as to impart the overall good taste of glutamic acid. Thus, the sodium chloride taste in a reduced-salt (sodium chloride) food or a drink can be improved and reduced food intake as a result of reduced saltiness or flavor can be prevented. Therefore, a low sodium chloride diet is provided for patients who require a restricted sodium diet, such as a hypertension patient, a cardiac disease patient, or a kidney disease patient. The reduced sodium diet is also useful for a healthy person who wishes to follow a low sodium chloride diet to prevent lifestyle-related diseases without lowering energy or other nutrient intake from meals.

### BRIEF DESCRIPTION OF THE DRAWINGS

FIG. 1 shows the results obtained when comparing the strength of the saltiness of reduced-salt (low sodium chloride) miso soup to which magnesium glutamate has been added.

FIG. 2 shows the results obtained when comparing the strength of the overall taste of reduced-salt (low sodium chloride) miso soup to which magnesium glutamate has been added.

FIG. 3 shows the results obtained when (intervention test) comparing the amount of food intake using a reference meal and a reduced-salt diet using the same menu over two weeks.

### DESCRIPTION OF THE PREFERRED EMBODIMENTS

The magnesium glutamate can be a salt of glutamic acid, and magnesium can be a bivalence metal ion. Generally it is obtained by a reaction of glutamic acid and a magnesium salt such as a magnesium hydroxide. The glutamic acid contains the optical D-isomer and an L-isomer, and the L-isomer can be more useful because the L-isomer is a component of biological proteins. The method of producing glutamic acid is not limited, and any method can be used whereby glutamic acid is obtained by hydrolysis of a natural protein from an animal or plant, or obtained by fermentation or a chemical synthesis. In particular, a fermentation method can be used which employs a glutamic acid production fungus with molasses as a raw material since it is safe and inexpensive. The magnesium glutamate can also be designated as MDG (Magnesium Di-Glutamate).

A composition that contains magnesium glutamate and enhances saltiness, but does not taste salty itself when combined with sodium chloride or used in food or drink is described. It can be given to a patient such as a hypertension patient or a cardiac disease patient who must restrict sodium chloride intake due to high blood pressure or arteriosclerosis, a patient who needs a restricted diet such as a kidney disease patient, or a healthy person who follows a low sodium chloride diet to prevent lifestyle-related diseases. The composition can also be used to improve the decrease in energy intake or other nutrient intake caused by a decrease in the intake amount of food or a drink that can occur when following a low sodium chloride diet.

A method for enhancing the salty taste of a food or a drink is also described, and is characterized by the addition of magnesium glutamate to a food or a drink that contains sodium chloride. The amount of the added magnesium glutamate can be 1 to 200 weight % relative to the weight of sodium chloride present in the food or drink, 5 to 100 weight %, or even 10 to 80 weight %. The concentration of the composition in accordance with the presently disclosed subject matter in a food or drink can be, although it depends on the concentration of sodium chloride in the food or drink, 0.05 to 2 weight % relative to the weight of the food or drink, and can also be 0.1 to 1 weight %.

The food or drink to which the composition can be added can be, for example, a seasoning such as miso, soy sauce, sauce, stock, dressing, mayonnaise, tomato ketchup, or the like; a kind of soup such as a miso soup, broth, clear soup, egg soup, wakame seaweed soup, potage, or the like; a kind of dipping broth or sauce for soba, udon, Chinese noodle, pasta, or the like; cooked-rice food such as a rice porridge, rice gruel, boiled rice soaked with tea, or the like; a livestock processed food such as a ham, sausage, cheese, or the like; a kind of snack food such as a potato chips, Japanese cracker, cookie, or the like; or a cooked food such as boiled food, deep-fried food, grilled food, curry, or the like.

The seasoning in accordance with the presently disclosed subject matter can contain sodium chloride and magnesium glutamate, and the weight ratio of the magnesium glutamate to the sodium chloride can be 0.5 to 100 weight %, 1 to 100 weight %, or even 5 to 100 weight %. Various kinds of additives suitable for foods or drinks such as an inorganic salt, acid, a kind of amino acid, a flavorful component based on or derived from nucleic acids, sugars, natural seasoning, spice, diluent, and the like can also be added, as necessary. Inorganic salts include potassium chloride, magnesium sulfate, or the like; and carboxylic acids include an ascorbic acid, fumaric acid, malic acid, tartaric acid, citric acid, or the like, and a salt thereof can be used. Amino acids which can be used as flavor enhancers include sodium glutamate, potassium glutamate, calcium glutamate, ammonium glutamate, glutamic acid, or the like. Each of these has umami taste (or flavor) from glutamic acid or special taste specific for each cation such as a sour taste from an ammonium salt. Flavorful components derived from nucleic acids include sodium inosinate, sodium gualylate, or the like. Sugars include sucrose, glucose, lactose, or the like.

The method for reducing sodium chloride in a food or a drink in accordance with the presently disclosed subject matter is characterized in that a portion of the sodium chloride present in the food or drink can be substituted with magnesium glutamate. The amount of magnesium glutamate added to the food or drink to be reduced in sodium chloride content can be 2 to 500 weight % relative to the weight of sodium chloride, 10 to 300 weight %, or even 10 to 100 weight %.

### EXAMPLES

The present invention will be explained in detail with reference to the following examples; however, the present invention is not limited to these examples.

### Example 1

Three types of miso soup were prepared: a normal miso soup, a miso soup having reduced sodium chloride (salt-reduced), and a miso soup having reduced sodium chloride and added sodium glutamate or magnesium glutamate. Sensory tests to determine the “strength of the salty flavor” and “overall strength of miso soup flavor” were performed. The amount of sodium chloride and additives in miso were calculated by a certified nutritionist in the Koshigaya Hospital attached to the medical department of the Juntenndo University. The miso soups were prepared such that the sodium chloride concentrations in the salt-reduced miso soups were the same. The components are shown in Table 1.

TABLE 1

| | | Relative |
|---|---|---|
| | | amount of |
| Sample | Composition | sodium |
| Reference 1 | Broth 120 ml | 1 |
| (normal miso soup) | Miso approx. 7 g | |
| Reference 2 | Broth 120 ml | 0.7 |
| (30% salt-reduced miso | Miso approx. 4.5 g | |
| soup) | | |
| Reference 3 | Broth 120 ml | 0.7 |
| (commericial salt-reduced | salt-reduced miso from | |
| miso soup from market) | market approx. 9.5 g | |
| Sample 1 | Broth 120 ml | 0.7 |
| (MSG + MDG) | Miso approx. 4.5 g | |
| | MSG 0.2 g | |
| | MDG 0.4 g | |
| | IMP 15 mg | |
| | GMP 2 mg | |
| Sample 2 | Broth 120 ml | 0.7 |
| (MDG) | Miso approx. 4.5 g | |
| | MDG 0.6 g | |
| | IMP 15 mg | |
| | GMP 2 mg | |

MSG: sodium glutamate
MDG: magnesium glutamate
IMP: inosinic acid
GMP: guanylic acid

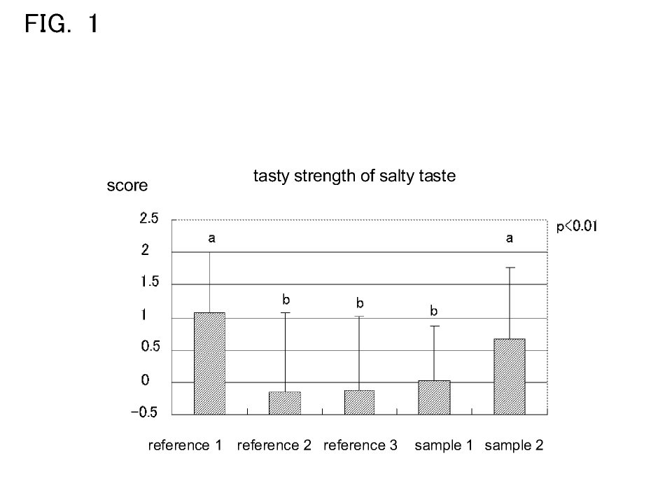

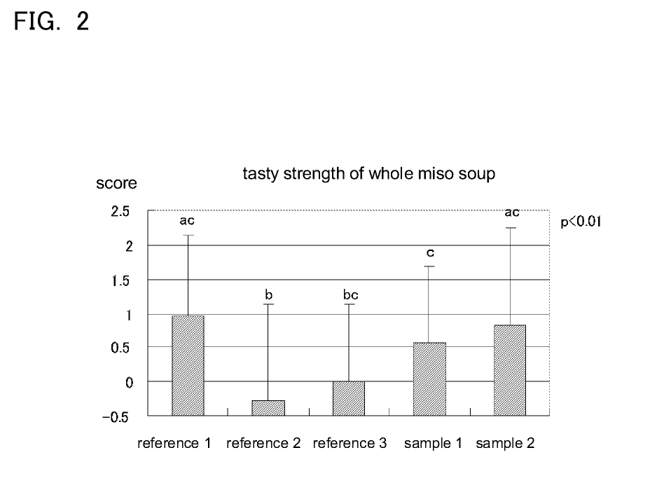

A scale with seven gradations, from −3 to +3, was used to evaluate the “strength of the saltiness” and “overall strength of miso soup flavor”. The test panel included 28 persons; 10 males and 18 females. The results are shown in FIG. 1 (strength of saltiness) and FIG. 2 (overall strength of miso soup flavor). A statistical significant difference was analyzed by a multiple comparison test using the Tukey's HSD test after variance analysis.

As shown in FIGS. 1 and 2, the “strength of saltiness” and “overall strength of miso soup flavor” markedly decreased in the samples with 30% reduced sodium chloride; however, a remarkable improvement was seen for both measures when MDG was added in sample 2. Furthermore, the effect was even larger when both MSG and MDG were added in sample 1.

### Example 2

A sensory evaluation test similar to that in Example 1 was performed, but using an experts' panel of six adult persons (one male and five females) who are in charge of research development at Ajinomoto Co., Inc. to confirm reproducibility of the results of Example 1. Sample solutions without broth were prepared as shown in Table 2 and tested. The sensory evaluation test results are shown in Table 3. A scale with 7 gradations, from 0 to 7, was used to evaluate the “strength of saltiness” and “overall strength of miso soup flavor”.

TABLE 2

| | | Converted |
|---|---|---|
| | | concentration of |
| | | sodium chloride |
| Sample | Composition | (weight) |
| Reference 1 | Hot water 440 ml | 0.624% |
| (30% salt-reduced) | Miso 16.39 g | (2.75 g) |
| | Sodium chloride 0.810 g | |
| Reference 2 | Hot water 440 ml | 0.625% |
| (MSG added) | Miso 16.39 g | (2.75 g) |
| | MSG 2.55 g | |
| | IMP 0.059 g | |
| | GMP 0.007 g | |
| Sample 1 | Hot water 440 ml | 0.625% |
| (MSG + MDG) | Miso 16.39 g | (2.75 g) |
| | Sodium chloride 0.398 g | |
| | MSG 1.274 g | |
| | MDG 1.274 g | |
| | IMP 0.059 g | |
| | GMP 0.007 g | |
| Sample 2 | Hot water 440 ml | 0.624% |
| (MDG) | Miso 16.39 g | (2.75 g) |
| | Sodium chloride 0.796 g | |
| | MDG 2.549 g | |
| | IMP 0.059 g | |
| | GMP 0.007 g | |

MSG: sodium glutamate
MDG: magnesium glutamate
IMP: inosinic acid
GMP: guanylic acid

TABLE 3

| | Reference 1 | Reference 2 | Sample 1 | Sample 2 |
|---|---|---|---|---|
| Strength of | 3.8 | 3.9 | 4.4 | 4.4 |
| saltiness | | | | |
| Strength of | 3.1 | 5.1 | 4.8 | 5.0 |
| overall | | | | |
| taste/flavor | | | | |

As shown in Table 3, the strength of the saltiness was significantly improved with the combined MDG and MSG in sample 1 and with MDG alone in sample 2. Thus, the strength of the saltiness is improved by adding MDG. The strength of overall taste with MDG alone in sample 2 was stronger to some degree than with the combined MDG and MSG in sample 1. The reason is that a metallic taste from the magnesium contributes to the overall strength of the taste/flavor when MDG is added alone. Thus, due to practical considerations for use as a seasoning, MSG should be added in combination with MDG because the metallic taste from the magnesium can be tempered.

### Example 3

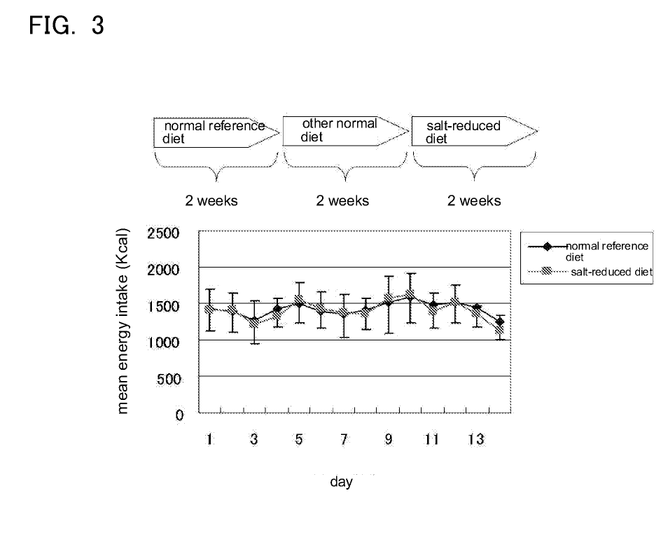

Diet plans were prepared using the same menus, but one plan contained a normal amount of sodium chloride (10 g/day), and a 30% salt-reduced plan contained on 7 g/day of sodium chloride. Four test subjects followed each diet for two weeks as follows. First, the four subjects followed the normal salt diet for two weeks, then the next two weeks followed a normal diet of different menus, and then the same four subjects followed the 30% salt-reduced diet for two weeks. Total energy intake (kcal) per day was calculated based on the amount of meal left uneaten, and the results are shown in FIG. 3.

In FIG. 3, the energy intake for each day is different day by day because each menu is different and each test subject has different food preferences. However, almost no difference in the energy intake was observed between the normal reference diet and 30% salt-reduced diet of the same menu. The results indicates that the food intake of the 30% salt-reduced diet was not changed compared with the normal reference diet and that the energy intake per meal can be maintained even when 30% of sodium chloride was reduced from the meal.

### INDUSTRIAL APPLICABILITY

The composition of the present invention can be used to reduce sodium chloride in foods or drinks without reducing the saltiness or flavor of the foods or drinks. Thus, this composition is useful for foods or drinks, especially for persons who wish or need to limit their sodium chloride intake.

The disclosures of all cited documents are hereby incorporated by reference in their entireties. The modes or examples of the invention can be modified or adjusted within the ambit of the entire disclosures (including the claims) of the present invention, and based on the technical scope of the invention. Moreover, various combinations or selections of the various disclosed elements are possible within the scope of the claims of the present invention.

What is claimed is:

1. A composition comprising magnesium glutamate which can enhance the saltiness of food or drink.

2. A method of enhancing the saltiness of a food or a drink comprising:
adding magnesium glutamate to a food or a drink comprising sodium chloride.

3. The method of enhancing the tastiness according to claim 2, wherein 1 to 200 weight % of magnesium glutamate is added relative to the amount of the sodium chloride present in the food or the drink.

4. A seasoning composition comprising sodium chloride and magnesium glutamate, wherein 0.5 to 100 weight % of magnesium glutamate is present in the composition relative to amount of the sodium chloride.

5. The seasoning composition according to claim 4, further comprising a component selected from a group consisting of sodium glutamate, potassium glutamate, calcium glutamate, ammonium glutamate, glutamic acid, a nucleic acid-derived component, and combinations thereof.

6. A method to reduce sodium chloride in a food or a drink comprising;
substituting a portion of the sodium chloride present in the food or a drink with magnesium glutamate.

7. The method according to claim 6, wherein the portion of magnesium glutamate is 2 to 500 weight % relative to the weight of sodium chloride.

8. A salt-reduced food or drink comprising the composition of claim 1.

9. A salt-reduced food or drink comprising the seasoning composition of claim 4.

10. A method for producing a salt-reduced food or drink, comprising:
A) preparing a composition comprising magnesium glutamate; and
B) adding said composition to a food and drink.

11. A method for using the salt-reduced food or drink described in claim 8, comprising administering said salt-reduced food or drink to a person selected from the group consisting of
A) a patient who must restrict their sodium chloride intake due to high blood pressure or arteriosclerosis,
B) a patient who must follow a restricted diet,
C) a healthy person who follows a low sodium chloride diet to prevent a lifestyle-related disease, and
D) combinations thereof
